(19) United States
(12) Patent Application Publication
(10) Pub. No.: US 2012/0245686 A1
(43) Pub. Date: Sep. 27, 2012

(54) System and Method for Attaching Soft Tissue to Bone

(76) Inventor: Maxwell Choongwon Park, Calabasas, CA (US)

(21) Appl. No.: 13/290,074

(22) Filed: Nov. 5, 2011

Related U.S. Application Data
(60) Provisional application No. 61/413,409, filed on Nov. 13, 2010.

(51) Int. Cl. A61F 2/08

(52) U.S. Cl. 623/13.14

(57) ABSTRACT

The ability to advance soft tissue into bone without externalizing it is considered an improvement for those knowledgable in the art of soft tissue fixation into bone. The above device/implant fixates soft tissue directly, then allows for fixation through a bony hole or aperture into a tunnel or void. It does not require interference fixation (eg. with a screw, or similar implant that fills a bony tunnel with soft tissue interposed). However, it does not preclude subsequent fixation that relies on interference fixation. The claims of this application disclosure for direct soft tissue fixation and subsequent bony fixation using the features of the described device/implant and insertor, as described in detail, are novel in this setting.

### RELATED APPLICATION

This application claims the benefit of priority of U.S. Provisional Application Ser. No. 61/413,409, filed on Nov. 13, 2010.

### BACKGROUND OF THE INVENTION

Current techniques for biceps tenodesis involve externalizing the tendon out of the body (eg., 2009/12011455 patent application; Arthrex technique guide for tenodesis screw). The newly described implant herein allows for in-situ fixation without externalizing the tendon, and therefore potentially significantly simplifies the procedure. Other systems have been described that do not necessarily externalize the soft tissue of interest, but technically require more steps, and in this context are more demanding to use. The described implant also addresses fixation in the setting of bone which provides only primarily cortical purchase (eg., proximal humerus). Notably, standard interference screw or interference fixation is not precluded from being used as supplemental fixation with this newly described implant and method of application—screw placement would potentially be much easier with the tendon already within the bony tunnel or void.

### SUMMARY

The following description is not intended to limit or define the claims.

The “Implant” or “Device” described herein provides 1) soft tissue purchase and 2) bony purchase, both essential and fundamental to the application of this invention. The implant or device provides intra-tendinous fixation which is unique in this setting; currently, interference screws are typically used for fixation into bone as the screw presses the soft tissue against adjacent bone within a bony tunnel (note therefore, this type of interference fixation does not directly or primarily secure the soft tissue). Therefore, the design element claims for this newly described implant are unique for the purpose of fixation into soft tissue. Importantly, it also necessarily relies on aperture-mismatch fixation (eg., toggle) that is intra-osseous. It does not require fixation around, or adjacent to (eg., interference fixation) the soft tissue of interest, which is a common method currently used.

Potential areas of application include: proximal biceps, coracoclavicular (CC) ligament reconstruction, collateral ligament reconstruction in large and small joints. Interference screw fixation can be problematic, particularly in the proximal humerus where the cancellous bone cannot be relied upon as readily (eg., in the setting of proximal biceps tenodesis with interference type fixation). For the proximal biceps, the newly described implant allows for arthroscopic in-situ fixation, without externalizing the tendon, which obviates the need for whip-stitching and suture management—this also allows for facilitating the maintenance of anatomic tendon length and tension.

### BRIEF DESCRIPTION OF THE DRAWINGS

FIG. 1 depicts one embodiment of the implant with essential features.

FIG. 2 shows another embodiment.

FIG. 3 shows an alternate embodiment.

FIG. 4 shows one embodiment of the distal insertor with fixed and mobile components in a cross-sectional view, disengaged from the implant.

FIG. 5 shows one insertor embodiment with: a handle, a primary shaft, and a movable component (the proximal aspect).

FIG. 6 shows a schematic of the implant, engaged to soft tissue, securely fixated to the deep side of cortical bone.

FIG. 7 shows pictures of a prototype embodiment, engaged to an insertor with fixed and movable aspects.

FIG. 8 shows a picture schematically depicting the insertor prior to advancement, creating an eccentric or rotational moment about the implant.

FIG. 9 shows an embodiment highlighting two of many different configurations with respect to “backstop” placement at the proximal middle region of the implant.

### DETAILED DESCRIPTION OF CERTAIN EMBODIMENTS

The following description of the described embodiments is exemplary in nature, and in no way does it limit the disclosure, its application, or uses.

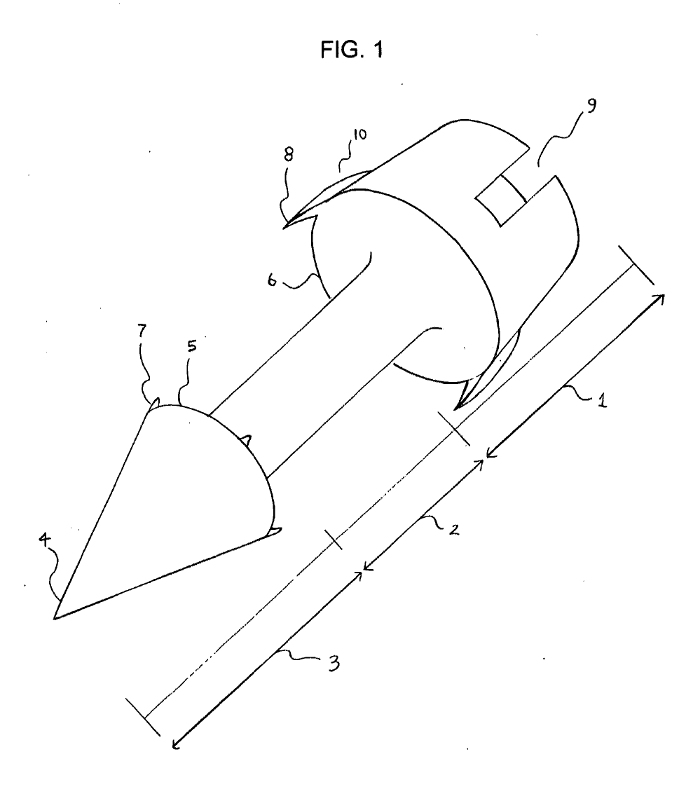

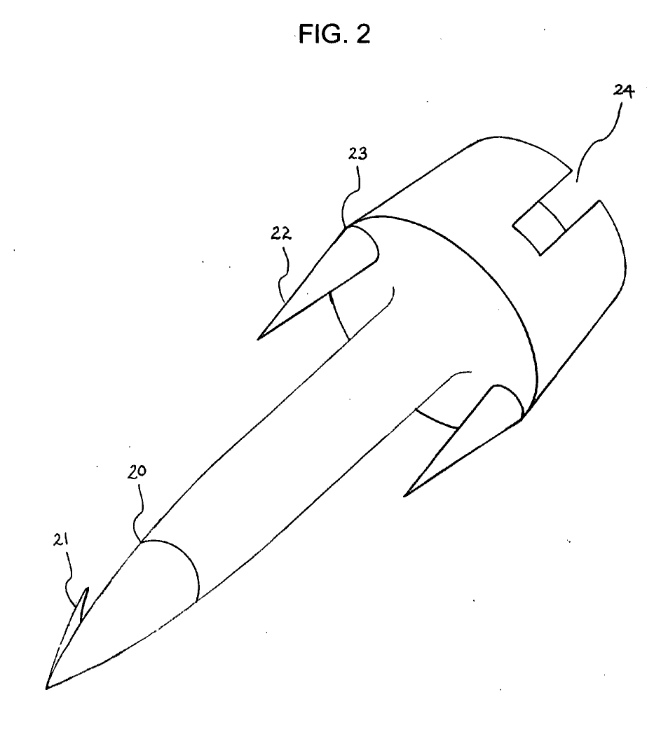

In FIG. 1, the implant has a proximal region or portion 1, a middle portion 2, and a distal portion 3. The distal end tip 4 pierces or otherwise engages the soft tissue and passes into said soft tissue—the distal tip 4 could have projections, prongs, barbs, or other similar projection that serve to help capture the soft tissue (not depicted in FIG. 1, but depicted in FIG. 2, 21). In one embodiment, a certain distance proximal to this distal-most end, is a “ridge” or lip 5, with none, one, or multiple prongs, barbs, or other appendages 7, emanating from said ridge 5 that capture the soft tissue with the emanations if present. The said ridge may be concentric (as drawn) or slanted. When more than one, the emanations may be symmetrically or asymmetrically placed, with uniform or variable lengths and or configurations. A certain distance distal to the proximal-most end is another ridge 6, with one or multiple prongs, barbs, backstops, or other appendages 8 that also help to capture the soft tissue—also, symmetrically or asymmetrically placed, with uniform or variable lengths and or configurations. The said ridge itself may be concentric (as drawn) or slanted. Therefore the soft tissue is wholly or in-part (with respect to the width or diameter of soft tissue) captured between the distal “ridge” 5 and proximal “ridge” 6 (eg. within the middle portion 2 of the implant). That portion between the said distal and proximal ridges constitute the middle portion of the implant. The proximal portion 1 contains a slot, hole, or other void 9 that is meant to mate, engage, or otherwise connect to an insertor instrument.

In FIG. 2, another embodiment is depicted that has no distal ridge present 20—the middle and distal portions are flush and continuous without an abrupt change in diameter or width between said portions. The distal end tip could have one 21 or multiple barbs, hooks, prongs, or other configuration that help(s) to capture or engage the soft tissue—these may be symmetrically or asymmetrically placed, with uniform or variable lengths and or configurations. From the proximal ridge, projections 22 that capture or otherwise engage the soft tissue can be present (single or multiple)—as shown, there are only 2 such projections. Notably, these projections may be placed in any geometrical configuration in relation to the ridge; they also may be flush to 23, or contained within, any outer circumference of the implant, or may protrude outward to a certain degree in relation to the circumference of the proximal portion (FIG. 1, 10). There is a slot, or other configuration that is meant to engage an insertor instrument.

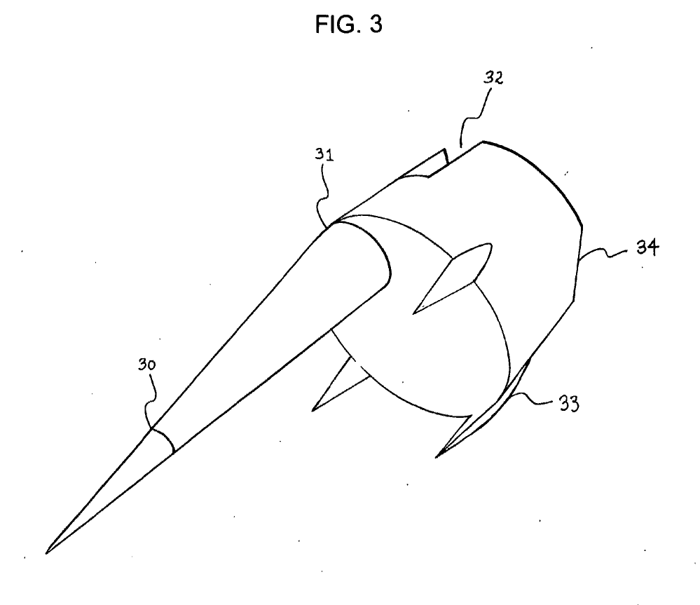

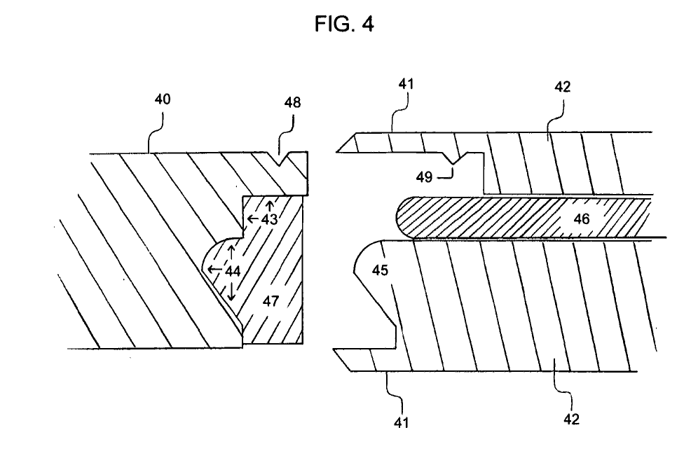

In FIG. 3, another embodiment is depicted that has no distal ridge 30. The middle and distal portions are placed off-center 31 in relation to the proximal region. The proximal ridge has multiple projections 33 meant to help capture soft tissue. There is a slot, or other void 32 meant to engage an insertor instrument. The proximal portion is depicted with a bevel or other configuration, meant to enhance how the implant engages the deep side of the bone. This bevel or other configuration may be placed anywhere along the proximal portion of the implant, including involving the said slot 32.

In FIG. 4, cross-sectional views of the proximal implant 40 and distal insertor 42, 46 are depicted. The implant has a notch 48, configured to mate or otherwise engage the distal insertor 49. The implant has a region 43 within a slot 47 meant to engage the distal shaft insertor 46. The implant has a region 44 within a slot 47 meant to engage the distal insertor 46 after the mobile or movable mate component 42 of the distal insertor is translated, rotated, or otherwise moved thus disengaging aspect 49 from aspect 48, the translation being more proximal to the shaft insertor 46. With the movable component 42 disengaged, the distal insertor 46 is freely movable to engage area 44 of said slot 47. With forcible advancement of distal insertor 46, when abutting, or otherwise engaging aspect 44 (which has an engageable off-center edge, lip, ridge, or the like) a rotational moment is created, thus rotating the implant. With movable component 42 in its primary position, a mate configuration 45 of the movable component, engages area 44 of said slot 47 to help maintain a certain rigidity between the insertor and implant. Aspects 41 are meant to circumferentially engage the proximal portion of the implant to connect, or maintain a certain rigidity and security between, the insertor and implant—cleat, spike, or snap aspect 49 also enhances this security when engaging mate-notch aspect 48 of implant 40. Aspects 41 may be of any variable length so far as to cover the entire implant, and may be of any configuration including tapered—this is in consideration for enhancing the ability to percutaneously advance the implant apparatus without the use of an additional cannula. With aspects 41 being long and tapered, the soft tissues will be relatively protected from the implant with its potential ridges and projections as it is advanced percutaneously. Once the implant apparatus is advanced to the soft tissue of interest percutaneously, the mobile component 42 may be translated proximally to allow the completion of the procedure. This consideration does not preclude the ability to use a standard cannula of appropriate length and diameter.

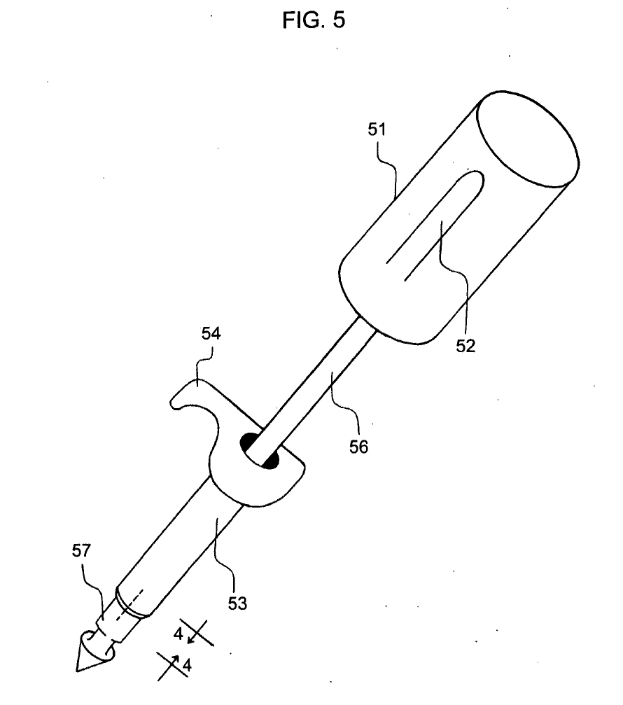

In FIG. 5, one embodiment of the insertor is depicted. The proximal aspect has a handle 51, with a groove 52; the groove, recess, or other configuration is meant to enhance grip for a particular functionality of the insertor, for example, push-in or screw-in functionalities. The insertor has a shaft 56 which corresponds to the distal shaft depicted in FIG. 4, 46. The movable component 53 corresponds to FIG. 4, 42 with its various configurations—movable component 53 can translate or rotate in relation to the shaft 56. The movable component has a projection or lip 54 that facilitates moving the movable component 53—this may be configured to accept one or multiple fingers of the operator. The implant 57 depicted at the distal end of the insertor is engaged to movable component 53; one embodiment relationship between implant 57 and movable component 53 is depicted in a sectional view at FIG. 5, 4-4 (which is seen in FIG. 4).

After the soft tissue is secured by the implant, a pilot hole (circular, ovoid, or slotted) is made at the desired location in bone for soft tissue fixation into bone with the implant.

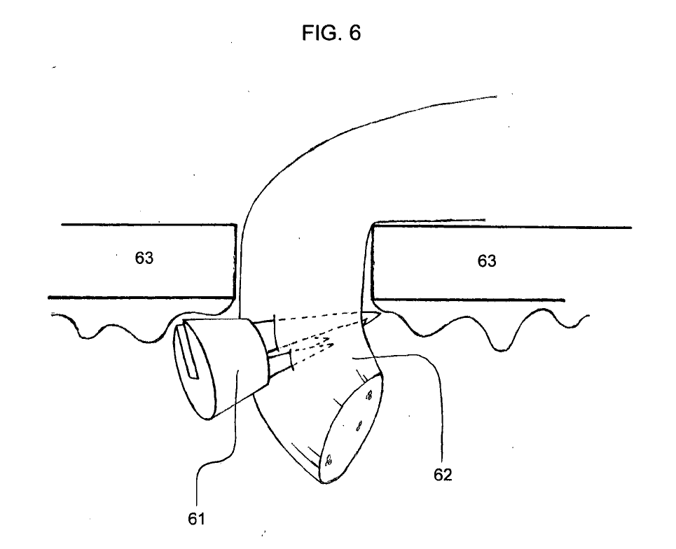

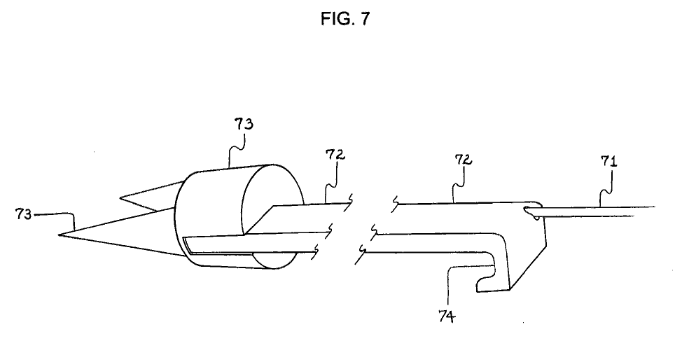

Once the proximal-most end is completely beneath the bone, a movable mate component 42, 53 of the insertor is pulled or rotated in the proximal direction via 54 (proximal to the tip of the shaft 46), leaving the fixed portion 46, 56 of the insertor in contact with the implant aspect 43 of slot 47; the movable portion of the insertor 42, 53 is now disengaged from the implant, including potential aspect 49 from aspect 48. With the movable portion of the insertor disengaged, the fixed portion (in relation to the handle) 46, 56 is forcibly advanced to rotate the implant over an “off-center” or eccentric center of rotation at aspect 44 of slot 47. This rotation of the implant creates a mismatch in the diameter of the implant 61 compared to the diameter of the pilot hole aperture, the diameter of the implant being larger. The implant is now secured to the deep surface of the cortical bone 63, along with the soft tissue 62 (FIG. 6).

For the proximal biceps, the biceps force vector will cause implant engagement on the deep side of the cortex, and the tendon diameter to compress and expand to a certain degree, further limiting pull-out. Cortical fixation is critical in this setting, and can be relatively difficult to purchase with interference screws alone.

FIG. 7 depicts an implant embodiment with insertor shaft 71 and movable component or mobile mate-component 72 both engaged to implant 73 within a space configured to mate both 71 and 72. The movable component 72 can be translated away from the implant using projection, lever, or handle 74 and is another embodiment of many compared to FIG. 4.

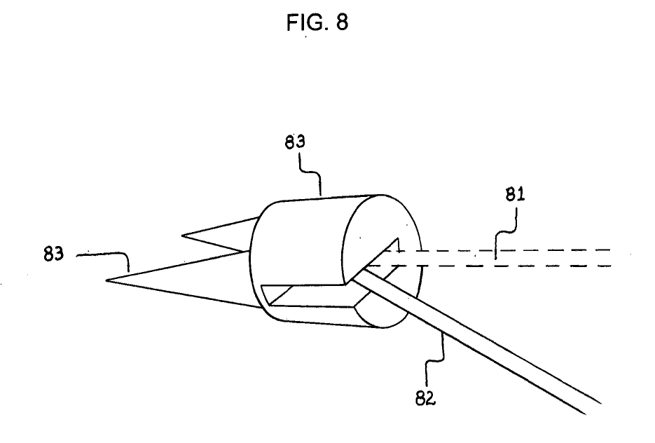

FIG. 8 depicts the insertor shaft 81 (dashed lines) moved to a secondary position 82 after movable component FIG. 7, 72 has been disengaged from implant 83. With the insertor at a secondary position off-center in relation to the implant 83, forcible advancement of the shaft 82 will create rotation of the implant deep to bone creating a mismatch in size between the implant and aperture of the bony hole into which soft tissue has been advanced and fixed by the implant.

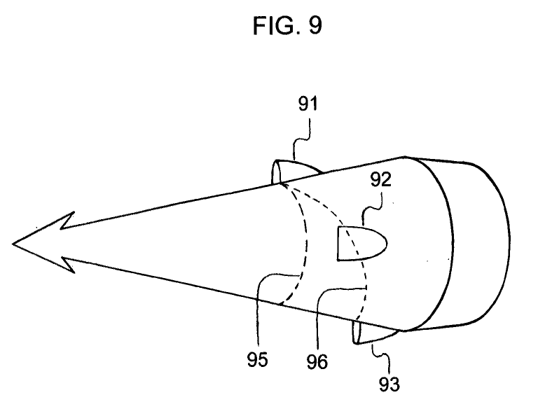

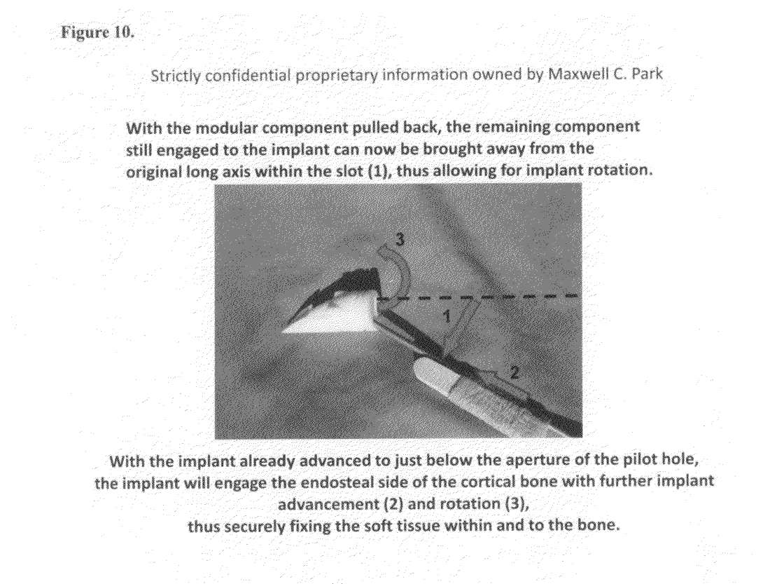

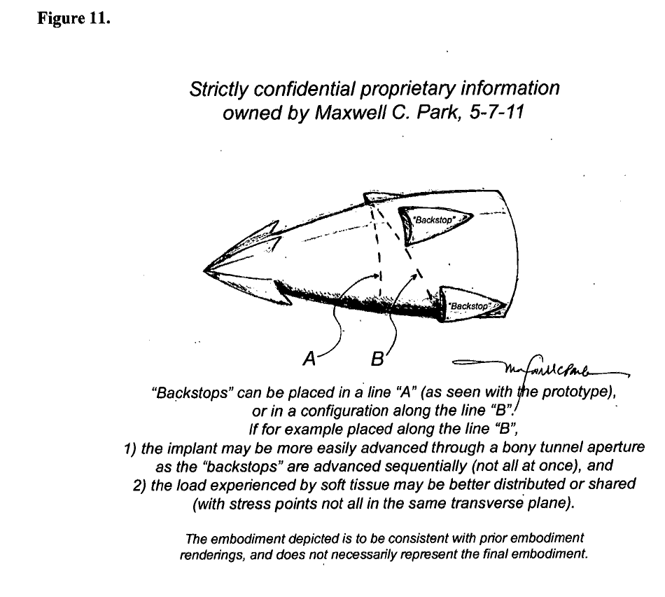

FIG. 9 depicts the possible placement for projections or “backstops” 91, 92, and 93 at the proximal region of the middle portion of the implant emanating outward from the implant. The configuration 96 may allow for easier advancement of the implant as the backstop emanations, projections, or protusions may be advanced sequentially distal to proximal, versus all at once (configuration 95), through the bony aperture.

In other embodiments the implant can be applied using an arthroscopic approach, or an open approach. The implant could have sliding, or non-sliding, sutures incorporated into its body, pre-existing or placed by the operator. The implant may be cannulated for other purposes (eg., sutures, venting, passing over guide-wire). Venting would allow healing factors and tissue to incorporate through and around the implant, thus enhancing fixation to soft tissue and bone.

Although the embodiments have been described in some detail by illustration and example for purposes of clarity of understanding, certain changes and modifications may be made without departing from the spirit or scope of the claims.

I claim:

1. A medical device comprising:
a proximal end,
a shaft emanating from the distal aspect of said proximal end,
and a distal end connected to an elongated implant, wherein the distal end is configured to disengage from the implant.

2. The medical device as in claim 1 wherein the distal end is connected to an implant via a mobile mate component.

3. The medical device as in claim 2 wherein the mobile mate component circumferentially incorporates or otherwise encompasses the insertor shaft, the movable component having a cannulation through which the insertor shaft passes, thereby allowing the movable component to slide or translate proximal and distal along the insertor shaft, and rotate about the insertor shaft.

4. The medical device in claim 2, the mobile mate-component distally being hollow having a certain outer and inner circumference, the outer circumference extending to any variable length including but not limited to the length of the implant, or any configuration, including and not limited to a tapered configuration.

5. The medical device in claim 4, having an inner circumference opening that is wider than the proximal implant so as to circumferentially encompass, mate, or otherwise engage the implant, thus stabilizing the connection between the shaft or distal insertor and said implant.

6. The medical device in claim 5, configured distally with a spike, cleat, elevation, snap, or other configuration on its inner circumference to mate the implant at the outer circumference of the proximal end said implant.

7. The medical device in claim 2, comprising a mobile mate-component that disengages the implant in its secondary translated, rotated, or otherwise moved position.

8. The medical device as in claim 1 wherein the shaft is configured to move in relation to the implant.

9. The medical device as in claim 8 wherein the shaft is configured to move from a first position in relation to the implant, to a second position in relation to the implant, wherein when the shaft is in the second position, the distal end of the shaft will engage the implant at another location within the implant.

10. The medical device as in claim 1 wherein the proximal end consists of a handle embodied to allow for screw-in or push-in advancement of the implant, or a combination of screw-in and push-in advancement.

11. The medical device as in claim 1 wherein the implant is configured to fixate soft tissue and into bone comprising:
A longer than wide including any combination of dimensions or sizes, generally cylindrical or rectangular structure, with a variable plurality of functional projections;
a distal region, middle region, and proximal region wherein:
the distal region is configured to pierce, screw, fixate, or otherwise engage soft tissue,
the middle region having a diameter or width less than or equal to the distal and proximal ends, is configured to engage or otherwise capture soft tissue,
the proximal region is configured to accept an inserting instrument, and
the regions are configured to engage bone.

12. The implant in claim 11 , the distal-most end comprising one or more tapered, sharp, pronged, threaded, serrated, beveled, notched, screw-like, self-tapping, cannulated, or dull-tipped, or any combination thereof, projection embodiments, with uniform or variable lengths, and symmetrically or asymmetrically placed.

13. The implant in claim 11, the proximal region with a flat, beveled, smooth, rough, cleated, spiked, or other configuration (single or multiple) meant to engage the deep endosteal side of cortical bone.

14. The implant in claim 13, the proximal-most end comprising one or more slots, bevels, notches, holes, and or other configurations to accept or mate an insertor instrument.

15. The implant in claim 14, said slots, bevels, notches, or holes being threaded or non-threaded to accept or mate an insertor instrument.

16. The implant in claim 13, with a notch or other mate configuration on its outer circumference to meet, snap, or otherwise engage a corresponding aspect of the distal insertor mate component inner circumference.

17. The implant in claim 14 with a proximal end configured with a lip, edge, ridge, or other mate-able configuration to create an off-center or eccentric rotation moment with forcible advancement of the insertor shaft, allowing for a diameter size mismatch between the implant and the underside bony aperture, the implant being larger.

18. The implant in claim 11, comprising a concentric or slanted ridge or lip at the distal middle region which prevent(s) soft tissue from disengaging from the implant or distal end.

19. The implant in claim 18, with a distal middle region consisting of projections emanating symmetrically or asymmetrically, from or around the ridge or lip region, that are either sharp, threaded, pronged, pointed, harpoon-like, barbed, tapered, blunt, tapered-sharp, tapered-blunt, beveled, beveled-sharp, beveled-blunt, or any combination of these descriptors, embodiments that project proximally (none, single, or multiple) with uniform or variable lengths, to capture, engage, fixate, or secure soft tissue.

20. The implant in claim 11, comprising distal and middle regions that are continuous, without a ridge or lip, being continuous straight or tapered.

21. The implant in claim 18, with the distal or middle regions configured concentrically in relation to the proximal end.

22. The implant in claim 20, with the distal or middle regions configured concentrically in relation to the proximal end.

23. The implant in claim 18, with the distal or middle regions configured eccentrically in relation to the proximal end.

24. The implant in claim 20, with the distal or middle regions configured eccentrically in relation to the proximal end.

25. The implant in claim 11, comprising a concentric or slanted ridge or lip at the proximal middle region, that prevent(s) soft tissue from disengaging from the implant or proximal end.

26. The implant in claim 25, with a proximal middle region consisting of projections or backstops emanating symmetrically or asymmetrically, from, or around, the ridge or lip region that are either sharp, threaded, pronged, pointed, harpoon-like, barbed, tapered, blunt, tapered-sharp, tapered-blunt, beveled, beveled-sharp, or beveled-blunt, or any combination of these descriptors, embodiments that project distally (none, single, or multiple) with uniform or variable lengths to capture, engage, fixate, or secure soft tissue.

27. The implant in claim 19 comprising projections that are placed either in line with each other along a circumferential line around, or concentric in relation to, the implant, or along a spiral or oblique line around the implant in their respective middle regions, distal and proximal.

28. The implant in claim 26 comprising projections that are placed either in line with each other along a circumferential line around, or concentric in relation to, the implant, or along a spiral or oblique line around the implant in their respective middle regions, distal and proximal.

29. The implant in claim 19 comprising protusions that project outward, orthogonal from the implant long axis, from the the primary implant, or are flush to, or contained within, the outer circumference of the implant in their respective regions, distal or proximal.

30. The implant in claim 26 comprising protusions that project outward, orthogonal from the implant long axis, from the the primary implant, or are flush to, or contained within, the outer circumference of the implant in their respective regions, distal or proximal.

31. The implant in claim 11, comprising a cannulation, eyelet, slot, hook, or other configuration to accept suture(s) either pre-existing with the implant, or suture(s) or wire(s) from an external source, for additional soft tissue fixation, or additional implant fixation strength to the deep side of cortical bone, or guiding the implant to a target tissue such as soft tissue or bony tissue.

32. The implant in claim 31, comprising a cannulation within the implant, running the length of the implant, or a portion of the implant (eyelet, venting), parallel or obliquely in relation to the long axis.

33. A method for attaching soft tissue to bone comprising drilling a pilot hole, attaching soft tissue to an elongated implant, inserting the implant containing or fixating soft tissue through the said pilot hole via an insertor, disengaging a mate-component of the insertor from the implant, moving the insertor shaft to a secondary position in relation to the implant, and advancing the insertor against the implant such that the implant rotates in relation to the deep-side of said pilot hole.

34. A method using the implant in claim 31, where a suture or guide-wire already passed through soft tissue and or into bone is used as a guide to the soft tissue and or bone, as the suture or wire passes through the cannulation or eyelet.

35. A kit comprising the medical device as in claim 1.

36. The kit of claim 35 further comprising a cannulated drill-bit and drill-bit guide pin.
